(19) United States
(12) Patent Application Publication
(10) Pub. No.: US 2017/0189391 A1
(43) Pub. Date: Jul. 6, 2017

(54) Protein kinase C inhibitor for treating triple-negative breast cancer

(71) Applicant: Macau University of Science ang Technology, Macau (CN)

(72) Inventors: Ying Xie, Macau (CN); Wenzhe Ma, Macau (CN); Wanjun Lin, Macau (CN); Jiajun Huang, Macau (CN); Zhongwen Yuan, Macau (CN); Shengling Feng, Macau (CN)

(21) Appl. No.: 14/985,416

(22) Filed: Dec. 31, 2015

(51) Int. Cl. A61K 31/4741; A61K 31/337

(52) U.S. Cl. CPC A61K 31/4741; A61K 31/337

(57) ABSTRACT

Protein kinase C plays important roles in TNBC development and could be a specific target. The in vitro anti-proliferative activity of PKC inhibitor chelerythrine on a panel of breast cancer cell lines was evaluated. Chelerythrine selectively inhibited the growth of TNBC cell lines over non-TNBC cell lines as demonstrated by cell proliferation assay and colony formation assay. The selective anti-proliferative effect of chelerythrine was associated with the differential apoptosis induction ability on breast cancer cell lines and induction of cell cycle arrest in some TNBC cell lines. By analysis of the expression levels of different PKC subtypes, the inventors found multiple PKC isozymes may mediate the selective activity of chelerythrine on TNBC cells. Finally, combination of chelerythrine and chemotherapy reagent taxol showed synergistic/additive effect on TNBC cell lines.

### FIELD OF INVENTION

This invention relates to a method of treating triple-negative breast cancer with a protein kinase C inhibitor, and in particular a natural benzophenanthridine alkaloid isolated from Chelidonium majus.

### BACKGROUND OF INVENTION

Breast cancer is the most common cancer for women worldwide, with an estimated 1.67 million new cases diagnosed and more than half million deaths in 2012. Clinically, based on the expression levels of estrogen receptor (ER), progesterone receptor (PR), and human epidermal growth factor receptor 2 (HER2), breast cancer is classified into subgroups of hormone receptor-positive, HER-2-positive, and triple-negative breast cancer. Triple-negative breast cancer (TNBC), characterized by the absence of ER/PR and lack of overexpression of HER2, represents approximately 10-15% of all breast cancers.

As TNBC does not respond to either hormonal therapy or anti-HER2 agents, standard chemotherapy is currently the mainstay of systemic medical treatment therefor. TNBC initially responds to conventional chemotherapy, however patients frequently have rapid relapses and currently there is no effective treatment thereafter. In addition, TNBC is more aggressive than other subtypes of breast cancer, which has great propensity to metastasize to the lungs and brain. So patients with TNBC usually have a poor prognosis and a shorter overall survival chance compared with other subtypes of breast cancer. A lot of new therapies targeting poly (ADP-ribose) polymerase (PARP), epidermal growth factor receptor (EGFR), angiogenesis, mammalian target of rapamycin (mTOR), proteasome, cyclin-dependent kinase (CDK), histone deacetylase (HDAC), Src kinase, Wnt signaling, and phosphatidylinositide 3-kinases (PI3K) have been actively investigated in clinical trials in patients with TNBC. But none of these efforts reaches expected results, and to date, not a single targeted therapy has been approved therefor. Therefore, new targeted therapies are in urgent need for patients with TNBC.

### SUMMARY OF INVENTION

In the light of the foregoing background, the present invention, in one aspect, is a method of treating triple-negative breast cancer, including administering an effective amount of a protein kinase C inhibitor.

In an exemplary embodiment of the present invention, the protein kinase C inhibitor is a benzophenanthridine alkaloid. In a further exemplary embodiment, the benzophenanthridine alkaloid is chelerythrine. In a further exemplary embodiment, the method further includes administering an effective amount of taxol.

According to another aspect of the present invention, it provides a pharmaceutical composition including a protein kinase C inhibitor admixed with a pharmaceutical carrier for treating triple-negative breast cancer.

In an exemplary embodiment of the present invention, the protein kinase C inhibitor is a benzophenanthridine alkaloid. In a further exemplary embodiment, the benzophenanthridine alkaloid is chelerythrine. In a further exemplary embodiment, the pharmaceutical composition further includes taxol.

In a further aspect, the present invention is a method of treating triple-negative breast cancer, including administering an effective amount of a protein kinase C inhibitor and taxol.

In an exemplary embodiment of the present invention, the protein kinase C inhibitor is a benzophenanthridine alkaloid. In a further exemplary embodiment, the benzophenanthridine alkaloid is chelerythrine.

### BRIEF DESCRIPTION OF FIGURES

The patent or application file contains at least one drawing executed in color. Copies of this patent or patent application publication with color drawing(s) will be provided by the Office upon request and payment of the necessary fee.

FIGS. 1A, 1B and 1C show that chelerythrine (CHE) selectively inhibits proliferation of TNBC cell lines. Four non-triple-negative breast cancer (Non-TNBC) cell lines (MCF7, ZR-75-1, SK-BR-3 and MDA-MB-453) and four triple-negative breast cancer (TNBC) cell lines (MDA-MB-231, BT549, HCC1937 and MDA-MB-468) were used to determine the growth inhibition effect of chelerythrine. FIG. 1A shows the dose effect of chelerythrine treatment (72 hours) on the proliferation of non-TNBC cell lines as compared with TNBC cell lines. The cell number at each chelerythrine concentration is represented as a percentage of control (treatment without chelerythrine). Average values are calculated from three independent experiments performed in duplicate (n=3). FIG. 1B shows the time course of chelerythrine treatment (5 μM) on the proliferation of non-TNBC cell lines as compared with TNBC cell lines. The cell number at each time point is represented as a percentage of control (treatment without chelerythrine). Average values are calculated from three independent experiments performed in duplicate (n=3). FIG. 1C shows the colony formation after treatment with chelerythrine (5 μM) for indicated times. Representative colony formation assay plates are shown, which are quantified by counting colony number (n=4). Data is shown as mean±SD.

FIGS. 2A and 2B show that chelerythrine differentially induces cell cycle arrest in TNBC cell lines. Four Non-TNBC cell lines and four TNBC cell lines as mentioned above were treated with chelerythrine (CHE, 5 μM) for 24 hours. FIG. 2A shows the representative cell cycle distributions analyzed by flow cytometry. FIG. 2B shows the percentages of the total cell population in the four different phases of cell cycle (Sub-G0/G1, G0/G1, S, and G2/M) that were determined using FlowJo software. Average values are calculated from three independent experiments (n=3). Data is shown as mean±SD.

FIGS. 3A, 3B, 3C, 3D, 3E, 3F and 3G show that chelerythrine selectively induces apoptosis in TNBC cell lines. Four Non-TNBC cell lines and four TNBC cell lines as mentioned above were treated with chelerythrine (CHE, 5 μM) for 24 hours. FIG. 3A shows the visualization of apoptotic morphological changes by fluorescent microscope with Hoechst 33258 staining. Reprehensive pictures are shown (400×). FIG. 4B shows the Western blotting analysis of apoptosis marker cleaved nuclear poly (ADP-ribose) polymerase (cPARP). FIGS. 3C, 3D and 3E show the representative contour diagrams of FITC Annexin V/PI flow cytometry of cells. Fractions of apoptotic cells were quantified. Average values are calculated from three independent experiments (n=3). FIGS. 3F and 3G show that TNBC cell line MDA-MB-231 was treated with chelerythrine (CHE, 5 μM) for 0, 6 and 24 hours. Apoptosis was analyzed by flow cytometry with Annexin V/PI staining. Representative contour diagrams and quantified data are shown. Average values are calculated from three independent experiments (n=3). Data is shown as mean±SD.

FIGS. 4A and 4B shows that Protein Kinase C, Alpha (PRKCA) is not overexpressed in all TNBC cell lines. FIG. 4A shows the quantitative real-time PCR analysis of PRKCA in the average of four non-TNBC cell lines (which are MCF-7, ZR-75-1, SK-BR-3 and MDA-MB-453 cell lines) as compared with four TNBC cell lines. Average values are calculated from three independent experiments performed in duplicate (n=3). Data is shown as mean±SD. P-values are determined by Student's t-test. ***, P<0.001. FIG. 4B shows the Western blotting analysis of PRKCA protein.

FIGS. 5A and 5B shows that multiple PKC isozymes may mediate the selective inhibitory activity against TNBC cells of chelerythrine. FIG. 5A shows the relative mRNA expression levels (loge) of 12 PKC isozymes in human breast cancer cell lines from Cancer Cell Line Encyclopedia (CCLE) database (n=56). The heatmap represents color-coded expression levels of differentially expressed PKC isozymes in human breast cancer cell lines. The color scale ranges from saturated blue for the minimum to saturated red for maximum. Average values in the graph are the mean value of all the non-TNBC cell lines (n=30) and TNBC cell lines (n=26). FIG. 5B shows the quantitative real-time PCR analysis of PKN1, PKN2, PRKCD, PRKCH and PRKCZ in the four non-TNBC cell lines as compared with the four TNBC cell lines. Average values are calculated from three independent experiments performed in duplicate (n=3). Data is shown as mean±SD. P-values are determined by Student's t-test. *, P<0.05; **, P<0.01; ***, P<0.001.

FIG. 6 shows that chelerythrine enhances chemotherapy activity of taxol. Four TNBC cell lines were treated with either chelerythrine (CHE, 2 μM) or taxol (Tx, 10 nM) only, or a combination of these two drugs (CHE, 2 μM+Tx, 10 nM). The cell number is represented as a percentage of control (no drug treatment). Average values are calculated from three independent experiments performed in duplicate (n=3). Data is shown as mean±SD. P-values are determined by Student's t-test. *, P<0.05; **, P<0.01; ***, P<0.001.

### DETAILED DESCRIPTION OF THE PREFERRED EMBODIMENTS

As used herein and in the claims, “comprising” means including the following elements but not excluding others.

One potential target agent for TNBC treatment is protein kinase C (PKC), which is a serine/threonine protein kinase family of enzymes and plays a critical role in several disease processes including cancer, diabetes, autoimmune diseases, heart failure, Parkinson's disease, Alzheimer's disease, and many other important human diseases. An inverse relationship between ER and PKC activity and abundance in human breast cell lines and tumors has been firmly established. PKC is also elevated in malignant versus normal breast tissue, and overexpression of PRKCA (PKCα) is associated with antiestrogen resistance and tumor aggressiveness. PRKCA is shown to be a central signaling node and therapeutic target for breast cancer stem cells, which share similar profile of cell surface markers with TNBC. Furthermore, PRKCA is directly associated with TNBC both in cell lines and in patient tumors.

All of the above findings imply that PKC plays important roles in TNBC and could be a potential specific therapeutic target agent for TNBC. However, no studies of the anti-cancer effect of PKC inhibitors specifically targeting TNBC have been reported. One of the most specific PKC inhibitors is chelerythrine, which is a natural benzophenanthridine alkaloid isolated from Chelidonium majus and possesses diverse pharmacological activities including potent anti-cancer and cytotoxic activities. Here, the inventor reported the selective anti-proliferative activity of chelerythrine against TNBC cell lines. Studies suggest that chelerythine or other PKC inhibitors can act as regimens for patients with TNBC tumors.

1. Materials and Methods

1.1 Reagents and Antibodies

Chelerythrine and taxol were purchased from Melonepharma (Dalian, China). Trichloroacetic acid (TCA), propidium iodide (PI), Hoechst 33258, DNase-free RNase A, triton X-100, crystal violet and sulforhodamine B (SRB) were obtained from Sigma Aldrich. Antibody sources were as follows: cleaved nuclear poly (ADP-ribose) polymerase (cPARP, Cell Signaling); PRKCA (BD Biosciences); β-actin (Sigma Aldrich); horseradish peroxidase-conjugated secondary antibodies (Jackson Laboratory).

1.2 Cell Culture

Breast cancer cell lines MDA-MB-231, BT-549, HCC1937, MDA-MB-468, MCF7, ZR-75-1, SK-BR-3 and MDA-MB-453 (Cell Bank of the Chinese Academy of Sciences, Shanghai, China) were cultured in 1640 medium (Gibco) supplemented with 10% FBS (Gibco) and 1% Pen Strep Glutamine (100X, 10,000 Units/ml penicillin, 10,000 mg/ml streptomycin and 29.2 mg/ml L-Glutamine) (Gibco).

1.3 In Vitro Cell Proliferation Assay (SRB Assay)

The anti-proliferative effects of tested agents on breast cancer cell lines were assessed by sulforhodamine B (SRB) colorimetric assay as previously described. Briefly, cells were seeded in 96-well plates in a volume of 100 ml/well at densities of 5,000˜40,000 cells per well. After overnight incubation at 37° C. in a humidified incubator with 5% CO₂, 100 ml medium containing agents (2 X indicated concentrations) were added. After 72-hour treatment, attached cells were fixed with 50 ml cold 50% (w/v) trichloroacetic acid (TCA) for 1 hour at 4° C. and then stained with 100 ml 0.4% (w/v) SRB. The absorbency at 515 nm was measured using SpectraMax 190 microplate reader (Molecular Devices) after solubilizing the protein-bound dye with 200 ml 10 mM Tris base solution (pH 10.5). The IC50 value was defined as the concentration required for a 50% reduction in cell growth.

1.4 Colony Formation Assay

Cells were treated with 5 mM chelerythrine for various periods of time. Cells were then washed with PBS, plated in drug-free medium in 6-well plates at densities of 2,000 cells/well and incubated for 7-10 days in the absence of drug. Colonies were stained with 0.2% (w/v) crystal violet in buffered formalin for 10 minutes. The number of colonies was counted.

1.5 Flow-Cytometric Analysis of Cell Cycle

Flow cytometric analyses were performed to define the cell cycle distribution 180 after treatment with 5 mM chelerythrine. Cells were harvested, washed twice with PBS, resuspended in 0.5 ml PBS (1,000,000˜2,000,000 cells/ml). Then 4 ml 70% ethanol was added and the mixture was kept at −20° C. for 2 hours to fix the cells. Cells were stained for total DNA content with a solution containing 20 mg/ml propidium iodide, 200 μg/ml DNase-free RNase A, and 0.1% triton X-100 in PBS for 30 minutes at room temperature. Cell cycle distribution was analyzed by flow cytometry (BD Bioscience). The percentages of the total cell population in the four different phases (Sub-G0/G1, G0/G1, S, G2/M) of cell cycle was determined using FlowJo software.

1.6 Flow-Cytometric Analysis of Apoptosis

Cellular apoptosis was analyzed with BD Annexin V: Fitc Apoptosis Detection Kit I (BD Bioscience) by flow cytometry. Briefly cells were plated in 6-well plates (100,000˜400,000 cells/well) and treated with chelerythrine. At the indicated time point, cells were harvested, washed twice with cold PBS, and resuspended in 1X Binding Buffer at a concentration of 1,000,000 cells/ml. 100 μl cells were transferred to 1.5 ml conical tube and 5 μl FITC Annexin V and 5 μl propidium iodide were added. The mixture was gently vortexed and incubated at room temperature for 15 minutes in the dark, followed by adding 400 μl 1x Binding Buffer to each tube. Cells were filtered and analyzed by flow cytometry (BD Bioscience) within 1 hour. Total apoptotic cells (FITC Annexin V positive) were counted.

1.7 Assessment of Cell Morphological Changes

Cells were plated in 12-well plates (80,000-300,000 cells/well) and then treated with 5 mM chelerythrine for 24 hours. After incubation, cells were collected, washed with PBS and stained with Hoechst 33258 (11.1 mg/ml) in buffered formalin solution containing 5.6% NP-40. Living and apoptotic cells were visualized through DAPI filter of fluorescence inverted microscope (Leica DM2500 Fluorescence Microscope) at ×400 magnification.

1.8 Retrieval of Gene Expression Data from CCLE Database

Cancer Cell Line Encyclopedia (CCLE) data on breast cancer cell lines was used to compare mRNA expression of TNBC and non-TNBC cells. The log₂ radio of TNBC cell lines (n=26) versus non-TNBC cell lines (n=30) was analyzed using GENE and Prism software.

1.9 Quantitative Real-Time PCR

Cellular mRNA was purified by binding to poly(dT) magnetic beads (Dynal) and reverse transcribed using SuperScript III (Invitrogen) as described by the manufacturer. Quantitative real-time PCR was performed in duplicates three times by using SYBR Green (Molecular Probes) on the ViiA™ 7 Real-Time PCR System (Applied Biosystems). Data was expressed as relative mRNA levels normalized to the eukaryotic translation initiation factor (EIF3S5 or TIF) expression level in each sample. The primer sequences can be obtained upon request.

1.10 Western Blotting

Protein samples were prepared by adding RIPA buffer with protease inhibitor cocktail (Roche) to cells and diluted in SDS-PAGE protein sample buffer. Samples were heated for 5 minutes at 95° C. before fractionation on SDS-polyacrylamide gels. The proteins were then transferred to Immobilon P (Millipore) and incubated with primary antibodies overnight at 4° C. The membranes were then washed with TBST and incubated with the appropriate horseradish peroxidase-conjugated secondary antibodies at room temperature. Proteins were visualized with SuperSignal West Dura Extended Duration Substrate or SuperSignal West Pico Chemiluminescent Substrate (Thermo Scientific).

1.11 Dual-Drug Combination Assay

Breast cancer cell lines were plated in 96-well plates (5,000-40,000 cells/well), treated with various concentrations of chelerythrine and taxol, either alone or in combination for 72 hours. Cell number was determined by SRB assay. The combination index (CI) score was calculated using Compusyn software. The effects of drug combination were determined based on the CI values: CI<0.9 demonstrating synergy between the drugs; 0.9<CI<1.1 demonstrating additive effect between the drugs; CI>235 1.1 demonstrating antagonism between the drugs.

2. Results

2.1 Chelerythrine selectively inhibits proliferation of TNBC cell lines.

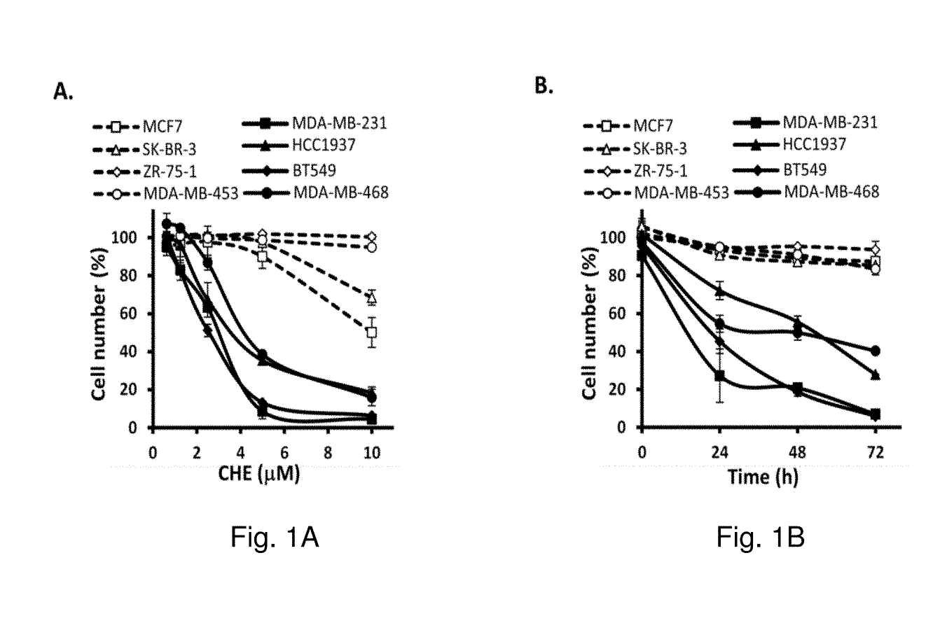

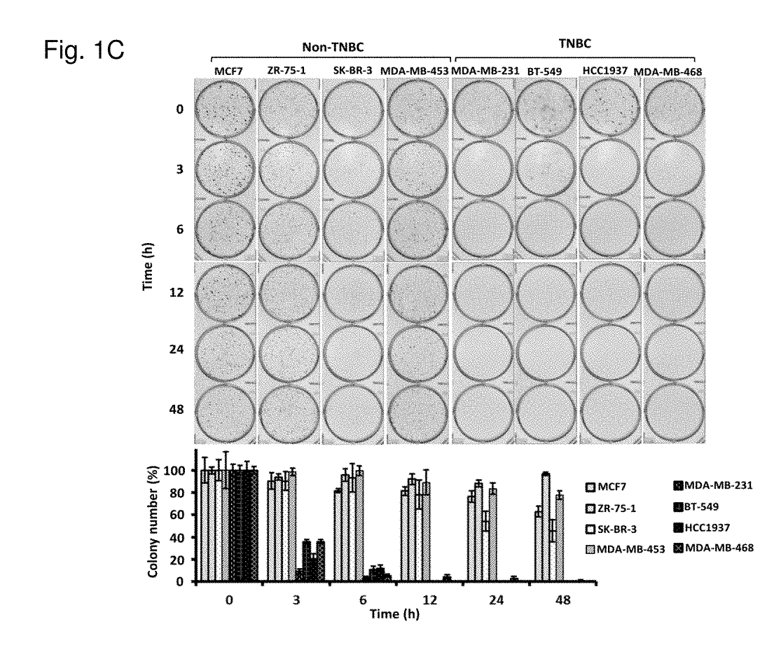

To test in vitro anti-proliferative activity of chelerythrine on breast cancer cells, four TNBC cell lines (MDA-MB-231, BT-549, HCC1937 and MDA-MB-468) and four non-TNBC cell lines (MCF7, ZR-75-1, SK-BR-3 and MDA-MB-453) were treated with a series of chelerythrine concentrations ranging from 0.625 mM to 10 mM for 72 hours. As shown in FIG. 1A and Table 1, chelerythrine inhibited the growth of TNBC cell lines dose-dependently, with IC50 values of 2.6 mM to 4.2 mM for MDA-MB-231, BT-549, HCC1937 and MDA-MB-468, respectively. On the contrary, the tested non-TNBC cell lines were relatively resistant to chelerythrine treatment on compared with the TNBC cell lines. The IC50 values of all of the four non-TNBC cell lines were greater than 10 mM, and two of the four cell lines, MDA-MB-453 and ZR-75-1, did not show any inhibitory effect even at the highest concentration tested.

When treated with 5 mM of chelerythrine, all of the four TNBC cell lines demonstrated significant reduction of cell growth at 24 hour and almost died out at 72 hour, while the four non-TNBC cell lines showed much less inhibition of cell growth at each time point, as shown in FIG. 1B.

From the result, it can be shown that the TNBC cells lines are more sensitive to chelerythrine treatment on compared with non-TNBC cell lines in a dose- and time-dependent manner The selective anti-proliferative activity of chelerythrine on TNBC cells were further confirmed by colony formation assay as shown in FIG. 1C. Colony formation ability of the TNBC cells was dramatically decreased after treatment with chelerythrine for 3 hours and almost no cells grew into colonies after 6 hours, while non-TNBC cells reserved largely of their colony formation ability even after treatment for 48 hours.

TABLE 1
Mean IC50 values (μM) of chelerythrine

| Non-TNBC | | | | TNBC | | | |
|---|---|---|---|---|---|---|---|
| MCF7 | ZR-75-1 | SK-BR-3 | MDA-MB-453 | MDA-MB-231 | BT-549 | HCC1937 | MDA-MB-468 |
| >10 | >10 | >10 | >10 | 3.0 | 2.6 | 3.6 | 4.2 |

2.2 Chelerythrine differentially induce cell cycle arrest in TNBC cell lines.

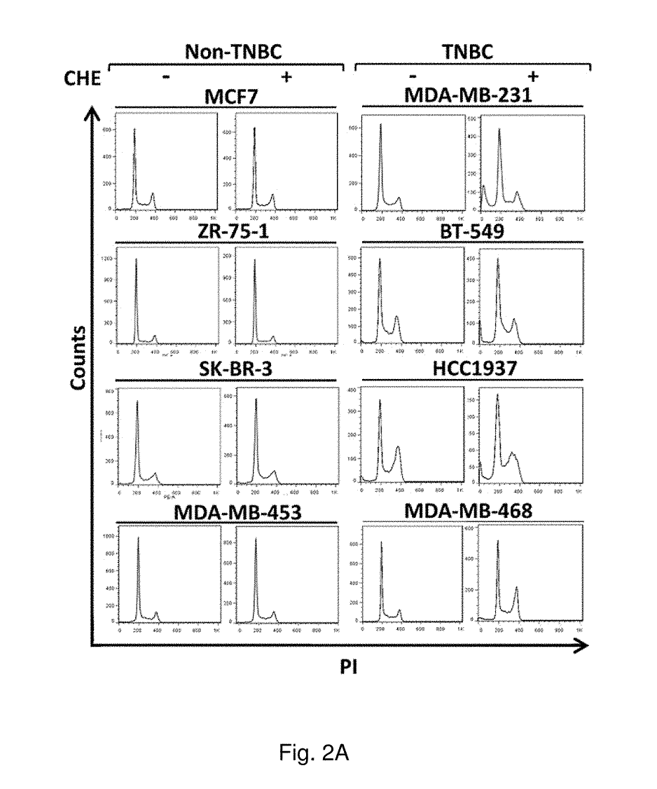

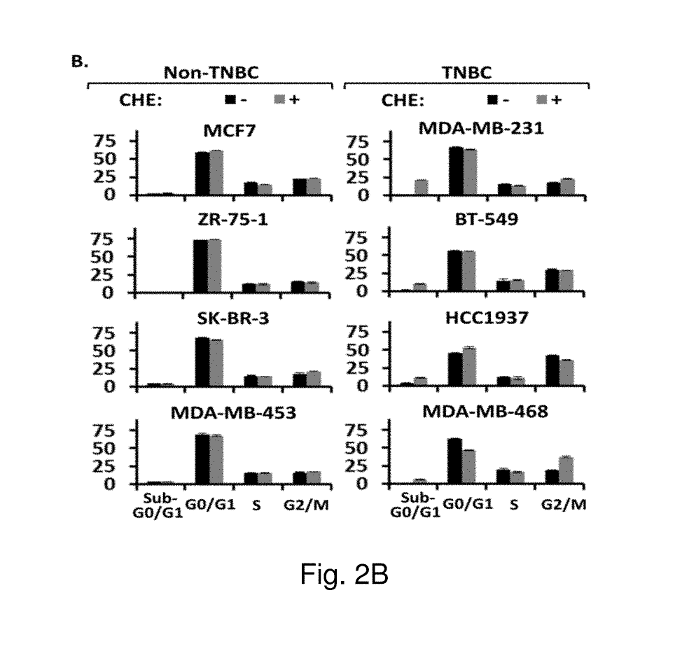

Cell cycle arrest is the key cellular event contributing to reduced proliferation, so the inventors first analyzed chelerythrine effects on cell cycle progression and the results were shown in FIGS. 2A and 2B. Consistent with the unnoticeable cell growth inhibition of 5 mM chelerythrine on non-TNBC cells, in which the results are shown in FIGS. 1B and 1C, the cell cycle distribution of these non-TNBC cells was basically unchanged with chelerythrine treatment (5 mM) for 24 hours. As for TNBC cell lines, the effect of chelerythrine on cell cycle progression was dependent on specific cell lines. There were no obvious changes in MDA-MB-231 and BT-549 cell lines. While the percentage of HCC1937 cells in G0/G1 phase increased from 45.1% to 52.9% upon chelerythrine treatment, there was a corresponding decrease of the number of cells in G2/M phase. Whereas MDA-MB-468 cells showed marked cell cycle arrest at G2/M phase. Chelerythrine differentially induced cell cycle arrest in TNBC cell lines, indicating other mechanisms existed for its selectivity on inhibition of TNBC cell growth. It is notable that a substantial proportion of nuclei had a sub-G0/G1 DNA content characteristic of apoptosis upon chelerythrine treatment in all TNBC cell lines, which implies induction of apoptosis could be the cause of selectively anti-proliferative effect of chelerythrine against TNBC cells.

2.3 Chelerythrine selectively induces apoptosis in TNBC cell lines.

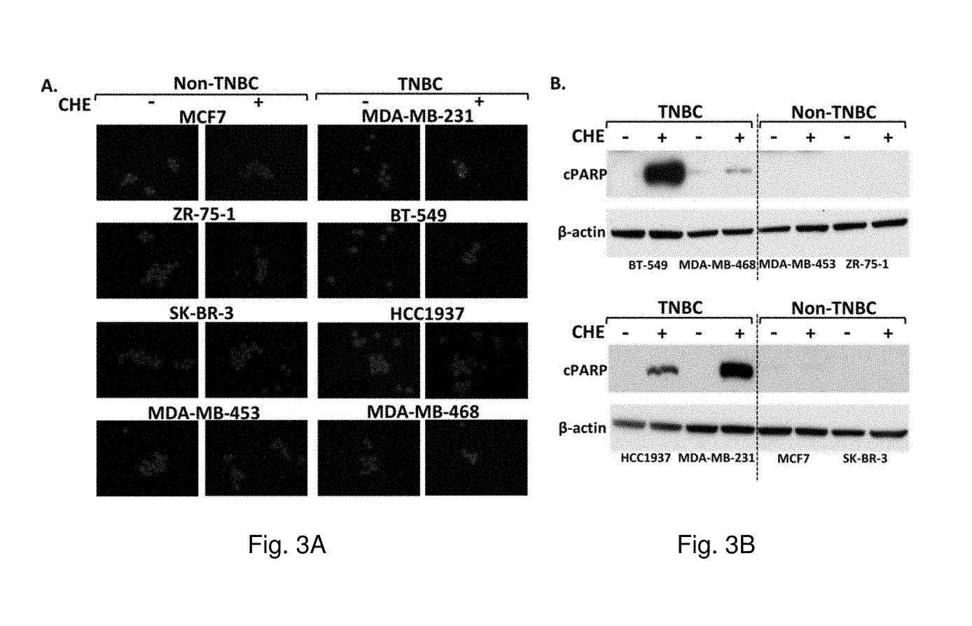

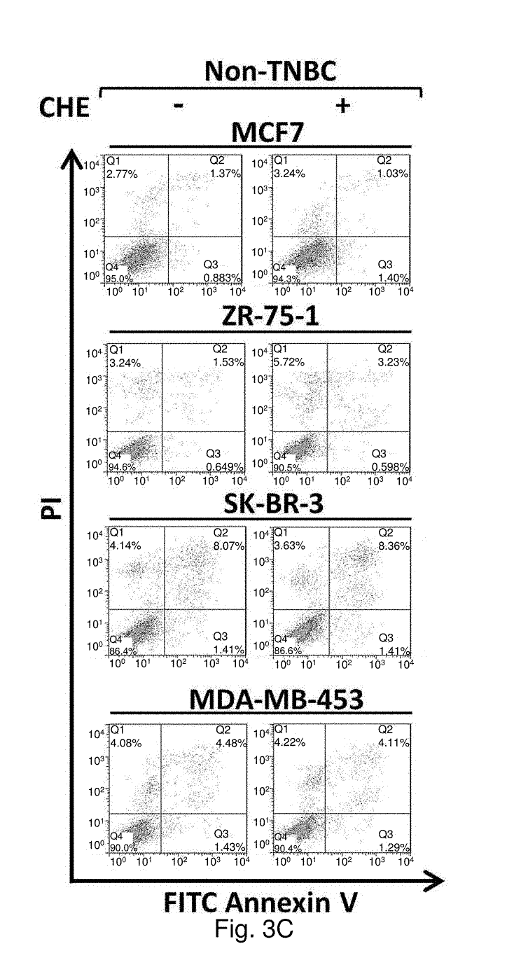

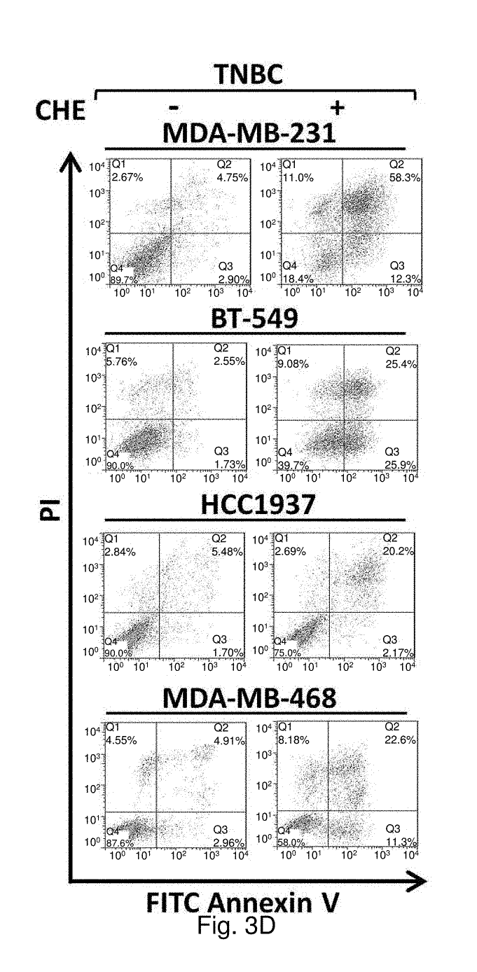

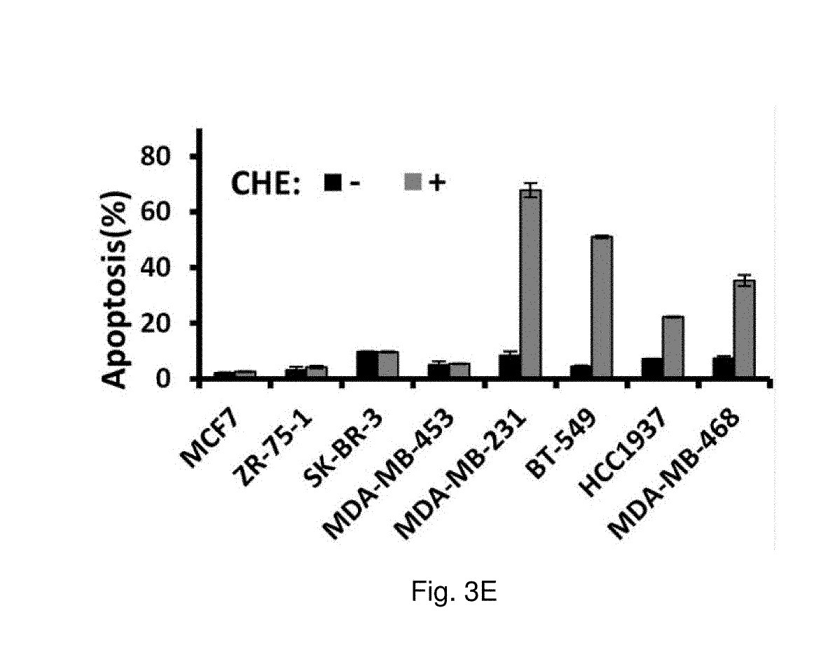

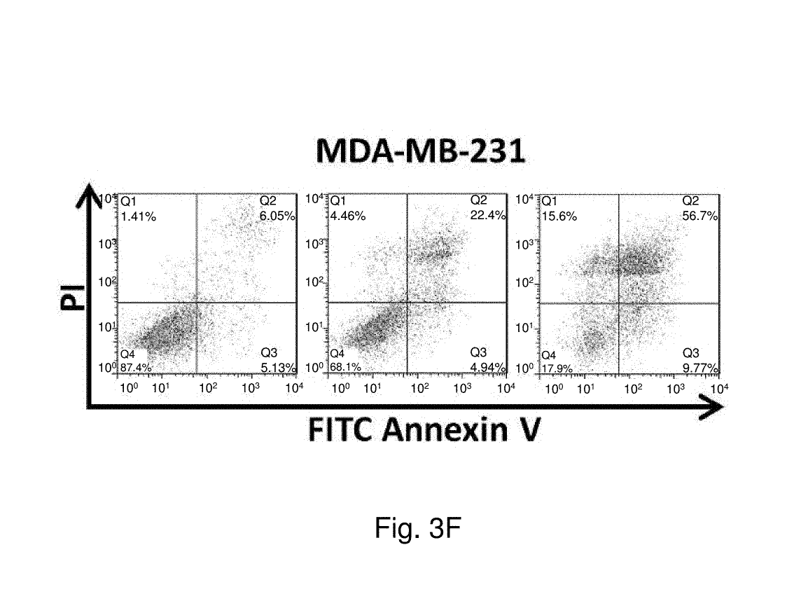

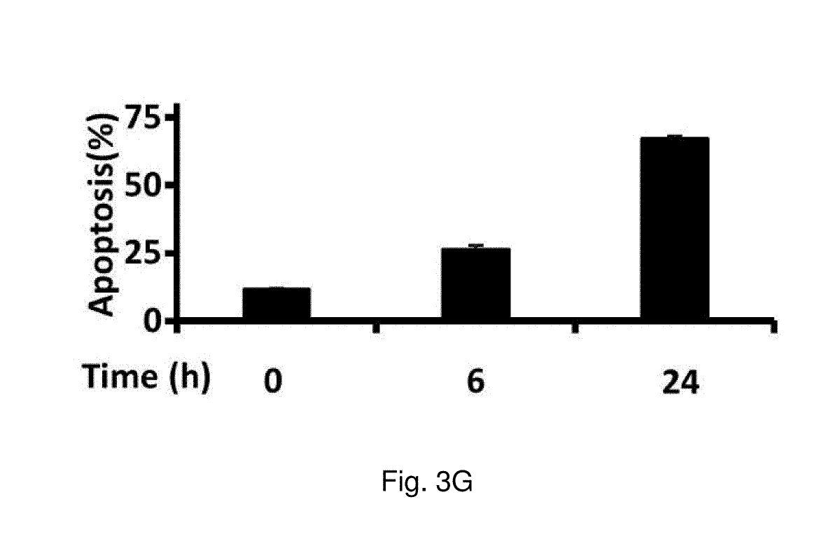

Chelerythrine has been reported to induce apoptosis in lymphoma cells, osteosarcoma cells, squamous cell carcinoma lines and melanoma cells. It was studied in this invention on whether apoptosis accounts for the selective anti-proliferative activity of chelerythrine on TNBC cells. As expected, chelerythrine indeed caused chromatin condensation and nuclear fragmentation, the typical morphological characteristics of apoptosis, in TNBC cells but not in non-TNBC cells as visualized by Hoechst staining as shown in FIG. 3A. This was further evidenced by the detection of the cleaved nuclear poly (ADP-ribose) polymerase (cPARP), a marker of apoptosis, in TNBC cell lines compared with non-TNBC cell lines after treatment with chelerythrine by western blotting as shown in FIG. 3B.

The apoptotic cell fractions were quantified by flow cytometry with annexin V and propidium iodide double staining after 24 hours of incubation with chelerythrine. FIGS. 3C, 3D and 3E show that the mean percentages of apoptotic cells after chelerythrine treatment of TNBC cell lines MDA-MB-231, T-549, HCC1937 and MDA-MB-468 were 67.8%, 51.0%, 22.2% and 35.3% respectively, whereas all of the four non-TNBC cell lines remained viable under the same treatment. The induction of apoptosis with chelerythrine treatment in TNBC cells was time-dependent as exemplified by MDA-MB-231 cells and was shown in FIGS. 3F and 3G. In short, the distinct apoptosis induction activity of chelerythrine on TNBC cells versus non-TNBC cells contributes to its selective anti-proliferative activity in breast cancer cells.

2.4 Multiple PKC isozymes may mediate the selective inhibitory activity of chelerythrine against TNBC cells.

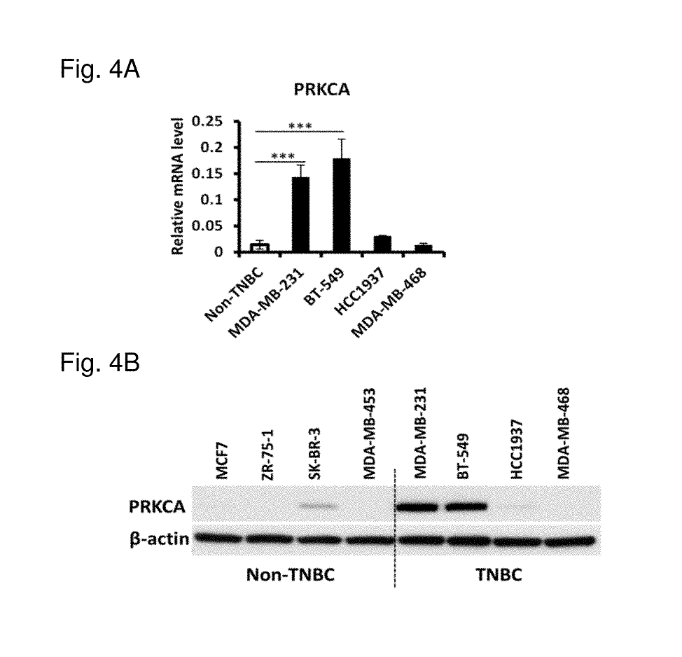

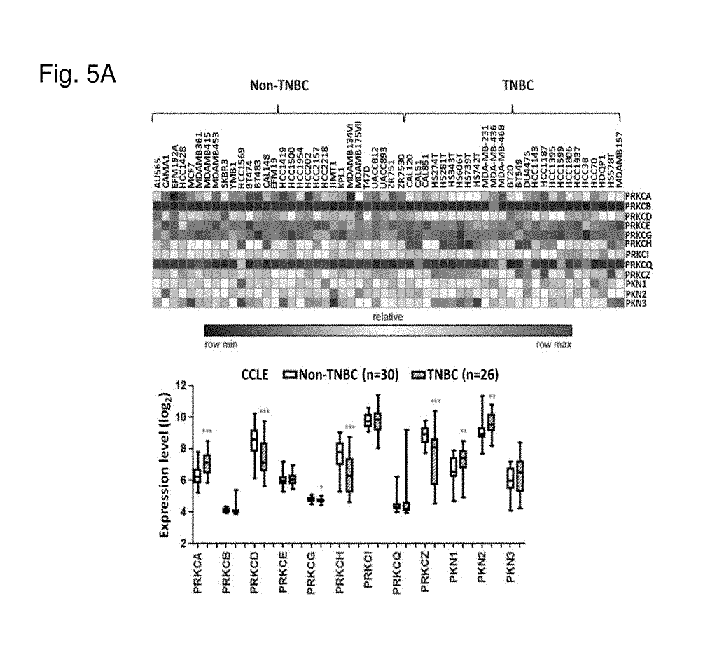

Chelerythrine is well known as a specific PKC inhibitor and several studies have suggested that PKC, especially the alpha isoform PRKCA, is a potential specific target for TNBC. To test this idea, the inventors measured the mRNA expression level of PRKCA in 8 breast cancer cell lines by quantitative RT-PCR. It was highly expressed in two of the TNBC cell lines, MDA-MB-231 and BT-549, and only marginally elevated in another TNBC cell line HCC1937 compared with non-TNBC cell lines as shown in FIG. 4A. This was further confirmed by western blotting analysis of the protein expression level (as shown in FIG. 4B) which is consistent with the previous study. PRKCA was not overexpressed in all TNBC cell lines and it was also detectable in the non-TNBC cells line SK-BR-3 (as shown in FIG. 4B), which implied that the selective anti-proliferative activity of chelerythrine on TNBC cells cannot be fully explained by expression levels of PRKCA.

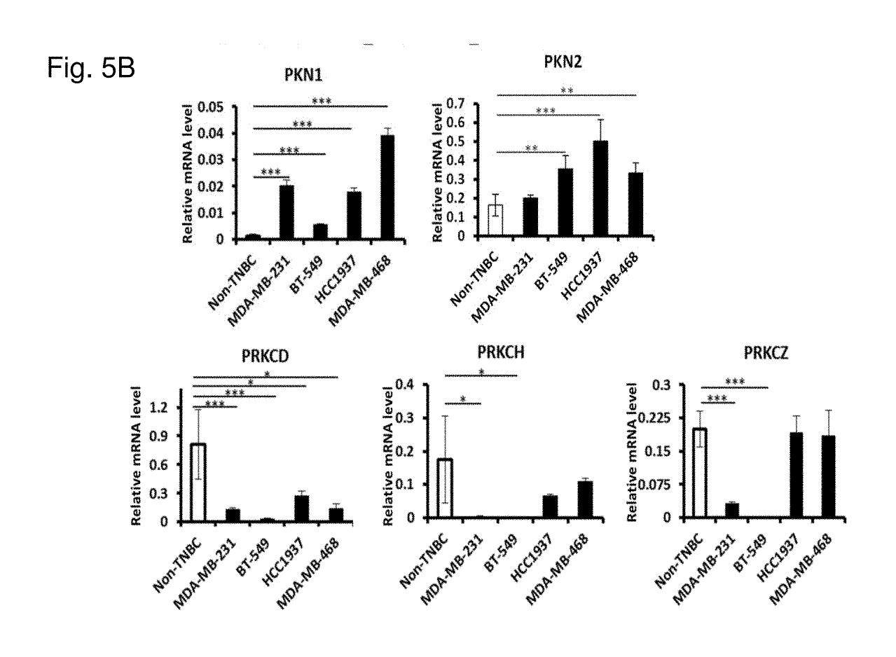

Since chelerythrine is an inhibitor of all PKC isozymes, the inventors reasonably expect that other PKC subtypes may also play roles in TNBC tumor biology. The inventors retrieved gene expression profile data of 12 PKC isozymes in 56 human breast cancer cell lines, including 26 TNBC cell lines and 30 non-TNBC cell lines, from the Cancer Cell Line Encyclopedia (CCLE) database. As shown in FIG. 5A, besides PRKCA, two other PKC subtypes, PKN1 and PKN2, were identified, which were significantly higher in the TNBC cell lines than in the non-TNBC cell lines.

The inventors analyzed the mRNA expression levels of these two PKC isozymes in the 8 human breast cancer cell lines by quantitative RT-PCR and the results were shown in FIG. 5B. PKN1 was significantly higher in all of the four TNBC cell lines compared with non-TNBC cell lines. PKN2 was also significantly higher in three TNBC cell lines including the two cell lines whose PRKCA were not significantly higher than non-TNBC cells. PKN1 was reported to be amplified in TNBC tumors and PKN2 was reported to play important roles in cell cycle progression and in cytokinesis, implying that these two PKC isozymes may cooperate with PRKCA to respond to chelerythrine for its anti-proliferative effects on TNBC cells.

Interestingly, the inventors also identified three PKC isozymes, PRKCD, PRKCH and PRKCZ, that were less expressed in TNBC cells compared with non-TNBC cells as shown in FIG. 5A. This was further confirmed by quantitative RT-PCR analysis of these three genes in the eight breast cancer cell lines as shown in FIG. 5B. PRKCD supports the survival of TNBC cells MDA-MB-231 by suppressing ERK1/2 pathway, while exerts proapoptotic effects in non-TNBC cell line MCF-7 and induces cell cycle arrest in another non-TNBC cell line SK-BR-3. So inhibition of PRKCD by chelerythrine may also contribute to the selective anti-proliferative effect on TNBC cells. Even the role of PRKCH and PRKCZ in cancer progression remain controversial, but in consistent with our findings, PRKCH was found decreased in invasive breast tumor tissues, which are mainly composed of TNBC cells, as compared with adjacent normal epithelium and in situ lesions. And low PRKCZ expression was reported to be associated with poorly differentiated breast cancer, the characteristic of TNBC tumors, with shorter patient survival. Taken together, the results of this study suggested that chelerythrine may exert selective anti-proliferative effect against TNBC cells through inhibition of multiple PKC isozymes.

2.5 Chelerythrine Enhances Chemotherapy Activity

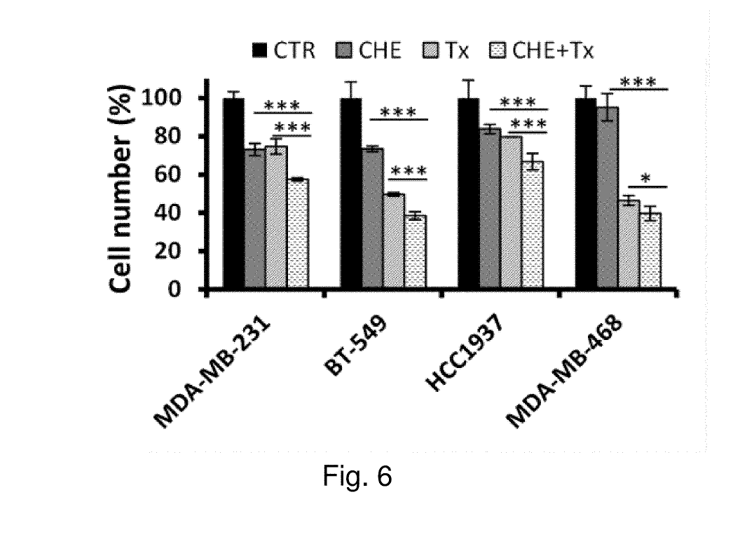

Currently there is no targeted therapy available for TNBC tumors and chemotherapy is still the standard regime for TNBC patients. The inventors, therefore, examined the effect of chelerythrine in combination with chemotherapy agent taxol. Treatment with dual drug combination significantly decreased cell proliferation than either drug given individually in all of the four TNBC cell lines and the results were shown in FIG. 6. Furthermore, dual treatment with both chelerythrine and taxol had either additive effect or synergistic effect as manifested by combination index (CI) values at ED50 ranging from 0.75190 to 1.13763 as shown in Table 2. The data suggest that combination of PKC inhibitors with chemotherapy agents may be benefit for patients with TNBC tumors.

TABLE 2
Combination index (CI) values at ED50 for
the combination of chelerythrine and taxol

| MDA-MB-231 | BT-549 | HCC1937 | MDA-MB-468 |
|---|---|---|---|
| 1.00098 | 1.13765 | 0.75190 | 1.08958 |

3. Discussion

TNBC is the most aggressive breast cancer with poor prognosis because of lack of effective and targeted approaches currently. Here, the inventors have shown that chelerythrine, a highly specific PKC inhibitor, selectively inhibit proliferation of TNBC cell lines both time- and dose-dependently, which was further demonstrated by dramatic difference of the colony formation ability between TNBC and non-TNBC breast cancer cell lines with chelerythrine treatment. It was further demonstrated that the selective anti-proliferative activity of chelerythrine on TNBC cells is resulted from substantial difference of apoptosis induction between breast cancer cell lines. Finally, chelerythrine significantly increased the cytotoxic effect of chemotherapy agent taxol against TNBC cell lines. Our data suggested that chelerythrine would be a promising regimen selectively targeting TNBC tumors which warranted further clinical development.

The PKC family consists of at least 12 isozymes that can be categorized into four subgroups specified by their divergent regulatory domains, while all isozymes share a highly conserved kinase/catalytic domain. Since chelerythrine interacts with the catalytic domain, it is active against all isozymes of PKC. Even though recent studies specified that PRKCA played important roles in TNBC tumors development, the data of this study showed that PRKCA is not detectable in some TNBC cell lines while overexpressed in non-TNBC cell line SK-BR-3. Besides PRKCA, PKN1 and PKN2 were overexpressed in most of the tested TNBC cell lines, and PRKCD, PRKCH and PRKCZ were significantly decreased as compared with non-TNBC cell lines (as shown in FIG. 5B). The data of this invention implies that multiple PKC isozymes may play complex roles in TNBC tumor development and the selective activity of chelerythrine on TNBC cells could be mediated through inhibition of multiple PKC subtypes simultaneously as a specific PKC inhibitor. Given the fact that the mechanism for each tumor cell to express multiple PKC isozymes remains elusive and rare PKC isozyme-specific inhibitors are available, it will be dependent on the clarification of the function of individual PKC isozymes to define the specific target of chelerythrine accounting for its selective activity observed in this study. On the other hand, the diverse pharmacological activities other than its PKC inhibitory function add more complexity to delineating the mechanism of chelerythrine on TNBC cells.

TNBC is a complex disease sharing 50-75% similarity with basal-like breast cancer. Efforts devoted to find targeted therapeutic approaches have been suffered from TNBC's heterogeneity. Detailed molecular characterization of TNBC is opening up new therapeutic possibilities. In this study, the inventors have delineated chelerythrine, a well-known inhibitor of protein kinase C, that it can selectively inhibit proliferation of TNBC cells in vitro. The results of this invention provide a rationale for future investigations into the subtle regulatory roles of PKC isozymes in TNBC tumors and the development strategies involving chelerythine or other PKC inhibitors for this hard-to-treat diseases clinically.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

What is claimed is:

1. A method of treating triple-negative breast cancer, comprising administering an effective amount of a protein kinase C inhibitor.

2. The method of claim 1 wherein said protein kinase C inhibitor is a benzophenanthridine alkaloid.

3. The method of claim 2 wherein said benzophenanthridine alkaloid is chelerythrine.

4. The method of claim 3 wherein said method further comprises administering an effective amount of taxol.

5. A pharmaceutical composition comprising a protein kinase C inhibitor admixed with a pharmaceutical carrier for treating triple-negative breast cancer.

6. The pharmaceutical composition of claim 5 wherein said protein kinase C inhibitor is a benzophenanthridine alkaloid.

7. The pharmaceutical composition of claim 6 wherein said benzophenanthridine alkaloid is chelerythrine.

8. The pharmaceutical composition of claim 7 wherein said pharmaceutical composition further comprises taxol.

9. A method of treating triple-negative breast cancer, comprising administering an effective amount of a protein kinase C inhibitor and taxol.

10. The method of claim 9 wherein said protein kinase C inhibitor is a benzophenanthridine alkaloid.

11. The method of claim 10 wherein said benzophenanthridine alkaloid is chelerythrine.
